(19) United States
(12) Patent Application Publication
(10) Pub. No.: US 2020/0215266 A1
(43) Pub. Date: Jul. 9, 2020

(54) SKELETAL MUSCLE STIMULATION FOR GLUCOSE CONTROL

(71) Applicant: Boston Scientific Scimed, Inc., Maple Grove, MN (US)

(72) Inventors: Vijay Koya, Blaine, MN (US); Bryan Allen Clark, Forest Lake, MN (US); Keith R. Maile, New Brighton, MN (US); Michael J. Kane, St Paul, MN (US); Jeffrey E. Stahmann, Ramsey, MN (US); Elizabeth Mary Annoni, White Bear Lake, MN (US)

(21) Appl. No.: 16/732,796

(22) Filed: Jan. 2, 2020

Related U.S. Application Data
(60) Provisional application No. 62/788,234, filed on Jan. 4, 2019.

(51) Int. Cl. A61M 5/172; A61M 5/168

(52) U.S. Cl. CPC A61M 5/1723; A61M 2005/14208; A61M 5/16831

(57) ABSTRACT

A system may include an input device configured to receive a signal indicative of elevated glucose in a patient, and an electrical stimulator configured to electrically stimulate at least one skeletal muscle. The system may further include a controller operably connected to the electrical stimulator. The controller may be configured for delivering a glucose therapy in response to the signal by controlling the electrical stimulator. The system may be configured to deliver the therapy in combination with other glucose therapies, and the therapy may be titrated based on a sensed parameter or patient feedback.

### CLAIM OF PRIORITY

This application claims the benefit of priority under 35 U.S.C. § 119(e) of U.S. Provisional Patent Application Ser. No. 62/788,234, filed on Jan. 4, 2019, which is herein incorporated by reference in its entirety.

### TECHNICAL FIELD

This document relates generally to medical devices, and more particularly, to systems, devices and methods used to provide glucose control.

### BACKGROUND

Diabetes is a metabolic disease that is prevalent throughout the world. Diabetes is commonly treated pharmacologically. However, the pharmacological approach currently lacks precision in glucose control and has significant side effects such as hypoglycemia, gastrointestinal problems, peripheral edema, body weight increase, pancreatitis, etc. Furthermore, the patient compliance to the pharmacological treatment plan is relatively low, such that many patients do not reach their glycemic goals, which can negatively impact the patient's health and health care cost. Therefore, there is a need for better glycemic control.

### SUMMARY

An example (e.g. “Example 1”) of a system may include an input device configured to receive a signal indicative of elevated glucose in a patient, and an electrical stimulator configured to electrically stimulate at least one skeletal muscle. The system may further include a controller operably connected to the electrical stimulator. The controller may be configured for delivering a glucose therapy in response to the signal by controlling the electrical stimulator to electrically stimulate at least one skeletal muscle.

In Example 2, the subject matter of Example 1 may optionally be configured such that the electrical stimulation is delivered using stimulation parameters selected to cause the at least one skeletal muscle to contract without causing discomfort in the patient.

In Example 3, the subject matter of any one or any combination of Examples 1-2 may optionally be configured such that the input device is configured with a user interface.

In Example 4, the subject matter of Example 3 may optionally be configured such that the signal is a user-provided signal received via the user interface.

In Example 5, the subject matter of any one or any combination of Examples 1-4 may optionally be configured such that the input device is configured to receive the signal from a glucose sensor system.

In Example 6, the subject matter of Example 5 may optionally be configured such that the glucose sensor system is configured to sense elevated interstitial or blood glucose.

In Example 7, the subject matter of any one or any combination of Examples 1-6 may optionally be configured such that the input device is configured to receive the signal from an activity sensor configured to sense an activity level of a patient, and the controller is configured to titrate the glucose therapy using the signal.

In Example 8, the subject matter of any one or any combination of Examples 1-6 may optionally be configured such that the input device is configured to receive the signal from a sensor configured to sense a temperature respiratory rate, heart rate, or blood pressure of a patient, and the controller is configured to titrate the glucose therapy using the signal.

In Example 9, the subject matter of any one or any combination of Examples 1-6 may optionally be configured such that the input device is configured to receive the signal from an electromyography (EMG) device, and the controller is configured to titrate the glucose therapy using the signal.

In Example 10, the subject matter of any one or any combination of Examples 1-9 may optionally be configured such that the controller is configured to control the electrical stimulator to electrically stimulate a motor nerve to contract the at least one skeletal muscle.

In Example 11, the subject matter of any one or any combination of Examples 1-10 may optionally be configured such that the controller is configured to control the electrical stimulator to electrically stimulate at least one skeletal muscle transcutaneously.

In Example 12, the subject matter of any one or any combination of Examples 1-10 may optionally be configured such that the controller is configured to control the electrical stimulator to electrically stimulate at least one skeletal muscle subcutaneously.

In Example 13, the subject matter of any one or any combination of Examples 1-12 may optionally be configured such that the at least one skeletal muscle includes a leg muscle of the patient.

In Example 14, the subject matter of any one or any combination of Examples 1-12 may optionally be configured such that the at least one skeletal muscle includes a back muscle of the patient.

In Example 15, the subject matter of any one or any combination of Examples 1-12 may optionally be configured such that the at least one skeletal muscle includes an abdominal muscle of the patient.

An example (e.g. “Example 16”) of a method may include delivering a glucose therapy to a patient having elevated glucose, where delivering the glucose therapy includes reducing glucose using an electrical stimulator to electrically stimulate at least one skeletal muscle.

In Example 17, the subject matter of Example 16 may optionally be configured such that the method further includes receiving a signal from a sensor configured to sense a level of glucose or a surrogate of glucose of a patient, and titrating the glucose therapy using the signal.

In Example 18, the subject matter of any one or any combination of Examples 16-17 may optionally be configured such that the glucose therapy further includes delivering insulin from an insulin pump.

In Example 19, the subject matter of any one or any combination of Examples 16-18 may optionally be configured such that the glucose therapy further includes ingesting oral insulin-releasing drugs.

In Example 20, the subject matter of any one or any combination of Examples 16-19 may optionally be configured such that the glucose therapy further includes percutaneously injecting insulin.

In Example 21, the subject matter of any one or any combination of Examples 16-20 may optionally be configured to include delivering the electrical stimulation in combination with a spinal cord stimulation.

In Example 22, the subject matter of Example 21 may optionally be configured such that the spinal cord stimulation includes therapy for pain reduction. In addition, the subject matter of Example 21 may optionally be configured such that the glucose therapy further includes the spinal cord stimulation.

In Example 23, the subject matter of any one or any combination of Examples 16-22 may optionally be configured to include delivering the electrical stimulation in combination with dorsal root ganglia stimulation.

In Example 24, the subject matter of any one or any combination of Examples 16-23 may optionally be configured to include delivering the electrical stimulation in combination with stimulation of targets in and around a portal vein.

In Example 25, the subject matter of any one or any combination of Examples 16-24 may optionally be configured to include delivering the electrical stimulation in combination with stimulation of targets in and around a hepatic artery.

An example (e.g. “Example 26”) may include a non-transitory machine-readable medium including instructions, which when executed by a machine operably connected to electrodes, cause the machine to perform any of the methods recited in Examples 17-25.

In Example 27, the subject matter of Example 26 may optionally be configured such that the instructions further cause the machine to receive a signal from a sensor configured to sense a level of glucose or a surrogate of glucose of a patient, and titrate the glucose therapy using the signal.

In Example 28, the subject matter of any one or any combination of Examples 26-27 may optionally be configured such that the instructions cause the machine to deliver electrical stimulation to one or more of a femoral nerve, a sciatic nerve, a saphenous nerve, a posterior femoral cutaneous nerve, a gluteal nerve, and an obturator nerve.

In Example 29, the subject matter of any one or any combination of Examples 26-28 may optionally be configured such that at least one of the electrodes is within a blood vessel to deliver electrical stimulation transvascularly.

In Example 30, the subject matter of Example 29 may optionally be configured such that at least one of the electrodes is within at least one of a femoral artery, a saphenous vein, or a femoral vein.

In Example 31, the subject matter of any one or any combination of Examples 26-30 may optionally be configured such that the instructions cause the machine to titrate the glucose therapy based on anticipated or scheduled dietary intake, anticipated or scheduled activity, or location of the patient.

In Example 32, the subject matter of any one or any combination of Examples 26-31 may optionally be configured such that the instructions cause the machine to titrate the glucose therapy based on a received input from the patient.

In Example 33, the subject matter of any one or any combination of Examples 26-32 may optionally be configured such that an intensity of the electrical stimulation is increased to increase a rate of glucose reduction.

In Example 34, the subject matter of any one or any combination of Examples 26-33 may optionally be configured such that the instructions cause the machine to deliver the glucose therapy on a schedule.

In Example 35, the subject matter of any one or any combination of Examples 26-34 may optionally be configured such that the electrical stimulation is delivered using stimulation parameters selected to cause the at least one skeletal muscle to contract without causing discomfort in the patient.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a skeletal muscle stimulation system to provide glycemic control.

FIG. 2A illustrates, by way of example, an embodiment of a stimulation system to provide glycemic control.

FIG. 2B illustrates, by way of example, intravascularly-delivered electrodes used to provide electrical stimulation to skeletal muscle targets.

FIGS. 2C-2D illustrate, by way of example, external electrodes used to provide electrical stimulation to skeletal muscle targets.

FIG. 3A illustrates, by way of example, a skeletal muscle target for electrical stimulation therapy; and FIG. 3B illustrates, by way of example, glucose control provided using electrical stimulation therapy of skeletal muscle targets.

FIG. 4 illustrates, by way of example, a method for glucose control using skeletal muscle stimulation.

FIGS. 5A-5B illustrate, by way of example, graphical representations of electrical stimulation signals used for skeletal muscle stimulation for glucose control.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. Further, the use of “and/or” may refer to “at least one of”, such that A and/or B refers to at least one of A or B, which may also be described as “A”, “B”, or “A and B”. The following detailed description is, therefore, not to be taken in a limiting sense, and the scope is defined only by the appended claims, along with the full scope of legal equivalents to which such claims are entitled.

Various embodiments described herein involve treatments that can provide glycemic control. For example, various embodiments may address patient compliance challenges as the treatment does not rely on a patient taking pharmaceuticals. The present subject matter provides for skeletal muscle stimulation, as either a non-invasive or invasive therapy, to improve glucose uptake and attain glucose control. In an embodiment, the skeletal muscle stimulation provides glucose therapy for patients with elevated glucose but not diabetes (e.g., patients with nondiabetic hyperglycemia). In another embodiment, the skeletal muscle stimulation provides glucose therapy for patients with diabetes (i.e., a diabetic therapy). The skeletal muscle stimulation provided for glycemic control may also avoid common complications associated with traditional therapy for elevated glucose including diabetic therapy in patients with diabetes.

Physiologic glucose levels in diabetic patients may be reduced by stimulating skeletal muscles or innervating neural targets that cause contractions of skeletal muscles. Skeletal muscles, when activated, can reduce blood glucose concentrations independent of insulin levels by increasing glucose diffusion into muscle cells. In one embodiment, skeletal muscles in a thigh area are stimulated for glucose control. In various embodiments, skeletal muscles in a back, calf or abdominal area are stimulated for glucose control. Other skeletal muscles can be stimulated for glucose control without departing from the scope of the present subject matter. The electrical stimulation is delivered using stimulation parameters selected to cause the at least one skeletal muscle to contract without patient discomfort, in various embodiments.

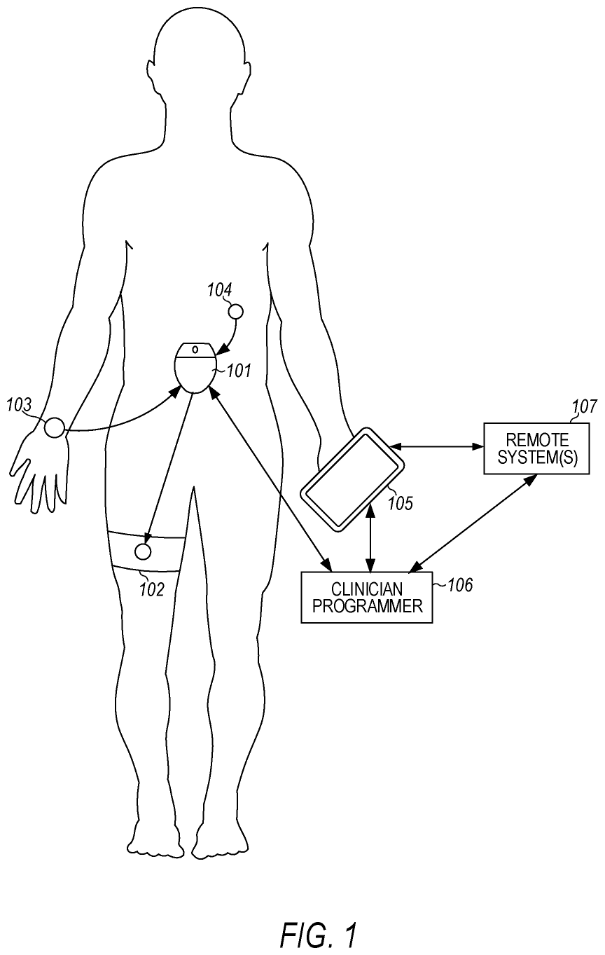

FIG. 1 illustrates, by way of example, an embodiment of a skeletal muscle stimulation system to provide glycemic control. The illustrated system 100 includes a pulse generator 101 that may be external to the patient or implanted, the pulse generator 101 configured to deliver electrical stimulation energy to the skeletal muscle target 102.

Various electrical stimulation therapies of skeletal muscles can be used to regulate glucose control. In one example, an electrical field is generated to cause contractions of a skeletal muscle. The muscular contraction increases energy demands on striated muscle cells, resulting in mobilization of GLUT4 transporters from within the cytoplasm to the cell membrane. The GLUT4 transporters permit diffusion of glucose from the blood stream down the concentration gradient and into the cells, effectively reducing glucose concentration in the blood stream of the patient.

The system 100 may include sensor(s), such as but not limited to an activity sensor 103 or a physiological sensor 104 such as glucose sensor. The depicted physiological sensor 104 is positioned near the stomach, as glucose sensors are usually positioned on the back of an arm, stomach or lower back, but the sensors can be positioned in different locations, and the location is the figure is simply an example. A number of sensors may be used, as disclosed throughout this disclosure. One or more sensors (implantable or non-invasive) may be integrated with or otherwise in communication with the pulse generator. Examples of sensors may include blood glucose sensor, interstitial fluid glucose sensors, and insulin sensors. These sensors may be used for closed-loop control. Other sensors may be used, such as sensors to detect glucagon, cortisol, progesterone/estrogen, norepinephrine/epinephrine, leptin, fatty acids/triglycerides, GLP-1, CCK, K+, Ca2+, Na+, Cl−, blood pH, interstitial fluid pH, activity levels (e.g. accelerometer data), respiratory rate, heart rate, blood pressure or a surrogate of blood pressure that may be used to quantify stress, hydration levels from blood flow via photoplethysmography or electrical bioimpedance, neural activity or evoked compound action potentials such as may be detected on parasympathetic or sympathetic nerve fibers. As will also be evident to one of ordinary skill in the art upon reading and comprehending this disclosure, the therapy control may be based, at least in part, on inputs from one or more of these sensors or from patient input. The system may be used to transform uncontrolled glucose levels into controlled glucose levels.

As illustrated in FIG. 1, the system may include one or more external devices, such as a patient device 105, a clinician programmer 106 and/or remote system(s) 107. The patient device 105 may function as a monitor, a remote control to control the therapy initiation, therapy termination, or therapy scheduling as discussed in more detail below. The patient device may also include a patient-facing interface for use by the patient to input data such as glucose levels, insulin dosages, activity, or other information. The patient-facing interface may be used by a patient to provide inputs such as meal start time and carbohydrates in meal. Other inputs may include exercise time, exercise intensity, exercise duration, sleep time, medication intake and time, alcohol intake, and/or menstruation information. A processor may use one or more of these input signals as an input to control stimulation parameters. The processor may use the time of day to determine normal daily patient trends as an input to control stimulation parameters.

The clinician programmer 106 may be used within a clinical setting to program modulation parameters in the pulse generator to cause the electrical energy to stimulate the appropriate target for skeletal muscle contraction. Also, the clinician programmer 106 may communicate with the patient device 105. In some embodiments, the patient device may be programmed by the clinician programmer. The patient device 105 and/or the clinician programmer 106 may communicate with remote systems(s) 107 that may be used to store or analyze patient-specific data or patient population data. Machine learning (also referred to as Artificial Intelligence or AI) may be implemented on patient-specific and/or patient population data to refine and improve upon the therapeutic response to various inputs. For example, AI may learn from the patient's typical daily activities over the course of days, weeks or months, and may use the patient's typical glucose levels, and/or dietary intake patterns as inputs for the automatically applied therapy. External system(s) may be used to program or update application(s) on the patient device 105 or clinician programmer 106.

The therapy system may operate in a manual mode, may operate in an automatic mode, and/or may operate in a semi-automatic mode. When the system is operating in a manual mode, the patient may be able to control the therapy using an external patient device 105 such as a handheld device. When the system is operating in an automatic mode, the system may deliver stimulation automatically, and in some embodiments may deliver closed-loop therapy based on sensor data. Sensed glucose levels may be used as a therapy input. For example, the target mean glucose level (e.g. 120 mg/dL) may be used as an input. Examples of high glucose thresholds may be within a range between 140 and 170 mg/dL. The present therapy may use patient activity as an input. The patient activity may be input by a person such as the patient or other user, or may be sensed by an activity sensor. Examples of activity sensors 103 may include an accelerometer, a gyroscope, a GPS sensor, a cardiovascular activity sensor, a respiratory sensor, or any other activity tracker or combination thereof. When the system is operating in a semi-automatic mode, an alert may be delivered to an external device (e.g. patient device 105 or clinician programmer 106), and the user (e.g. patient or clinician) may choose whether to take action on the alert.

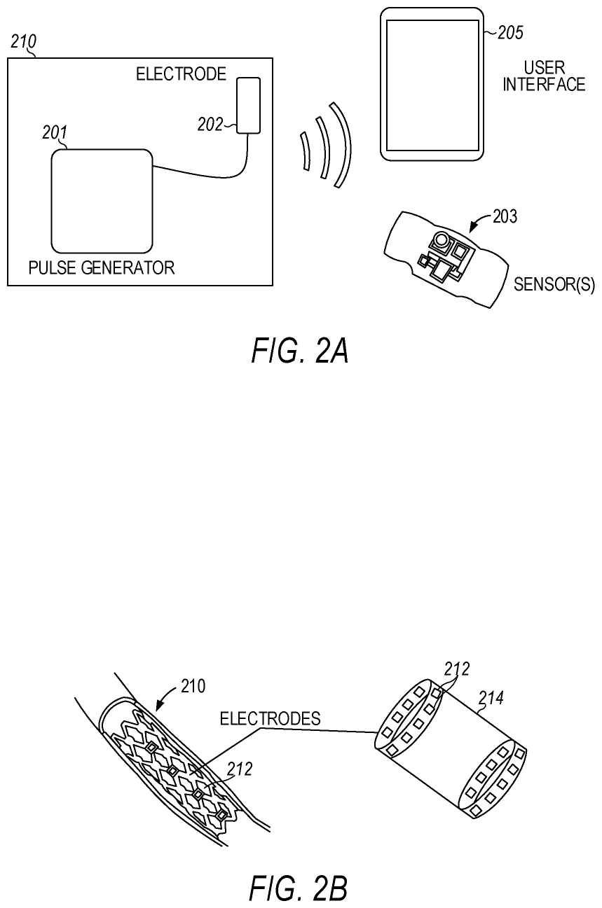

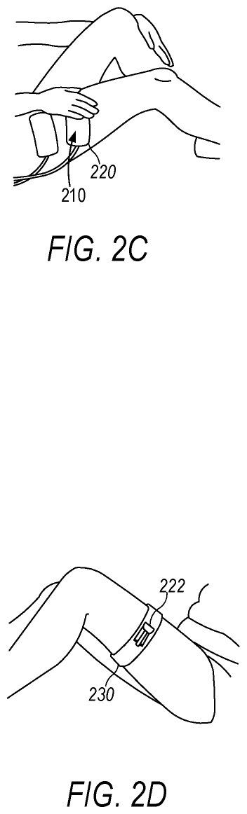

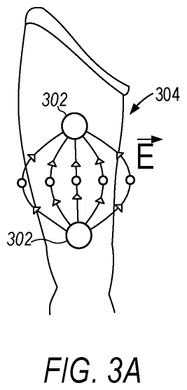

FIG. 2A illustrates, by way of example, an embodiment of a stimulation system to provide glycemic control. The system 210 may include one or more electrodes 202 at or near the target tissue electrically connected to one or more pulse generators 201. In various embodiments, the one or more pulse generators 201 generate an electric field in the target tissue of skeletal muscle using the one or more electrodes 202 to reduce blood glucose concentration. Optionally, the system additionally includes one or more physiological sensors 203 used to indicate therapeutic efficacy, assist with chronotherapy, or provide feedback for closed loop therapy, among other applications. Optionally, the system additionally includes a user interface 205 used to set or change stimulation and provide notifications such as battery life, glucose level, or other physiological sensor output. In some embodiments, the electrode and pulse generator may be implanted within the target tissue or within a vessel adjacent the target tissue. In some embodiments, the electrode and pulse generator may be placed externally to the patient on or around the area including the target tissue. In some embodiments, the electrode may be implanted in or near the target tissue, and pulse generator may be placed externally to the patient.

FIG. 2B illustrates, by way of example, intravascularly-delivered electrodes 212 used to provide electrical stimulation to skeletal muscle targets. In various embodiments, the electrodes 212 are arranged on a lead or cuff 214 and are placed (via catheter or other delivery mechanism) within a vessel 210 in a region adjacent targeted muscle tissue. In one embodiment, the electrodes are placed in a femoral vein for stimulation of skeletal muscle in the thigh. Electrodes may be placed in other vessels without departing from the scope of the present subject matter. A plurality of electrodes 212 may be outwardly facing from the cuff 214 toward targeted neural or muscle tissue. According to some embodiments, current delivered to each of the electrodes may be independently controlled. The lead may be delivered laparoscopically, percutaneously or surgically. A percutaneous procedure refers to a process for pacing the lead through skin and other tissue into position near the targeted neural tissue.

FIGS. 2C-2D illustrate, by way of example, external electrodes 222 used to provide electrical stimulation to skeletal muscle targets. In FIG. 2C, the electrodes 222 are attached to the skin of a patient adjacent target muscle tissue using an adhesive pad 220. The external electrodes 222 are adhered to the leg to generate an electric field in the thigh, in an embodiment. In FIG. 2D, the electrodes 222 are held in place near the skin of a patient adjacent target muscle tissue using an elastic band or strap 230. Other types of materials may be used to adhere the electrodes without departing from the scope of the present subject matter. In some embodiments, the system includes a handheld device.

In various embodiments, the electrodes may be implanted in the target tissue and the pulse generator or power supply is external to the patient. Various embodiments use a power transfer circuit to provide power from an external source to produce an electric, magnetic or electromagnetic field at the electrode (battery-less implant) in the target tissue. In other embodiments, an external source is used to recharge an implanted battery at the electrode for use in producing the field in the target tissue.

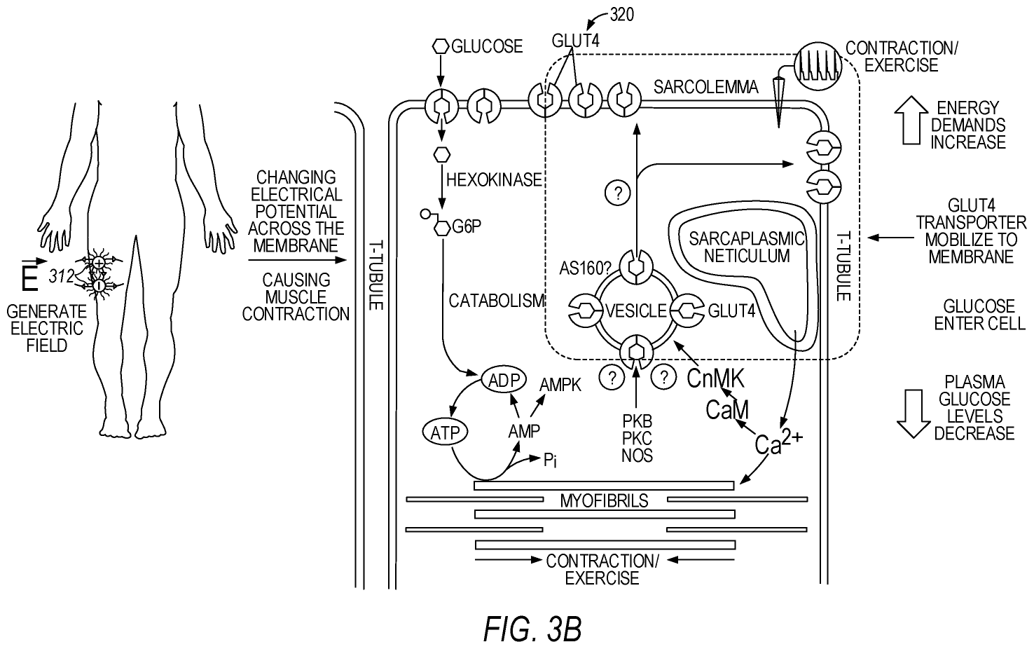

FIG. 3A illustrates, by way of example, a skeletal muscle target for electrical stimulation therapy. In the depicted embodiment, electrodes 302 have been implanted in a thigh region 304 of a patient, and electrical current is applied to create an electric field in the target tissue of leg muscle. FIG. 3B illustrates, by way of example, glucose control provided using electrical stimulation therapy of skeletal muscle targets. An electric field is generated in the thigh region 314 using electrodes 312. By changing electrical potential across a membrane, the leg muscle is forced to contract which increases energy demands of the leg muscle. The increased energy demand mobilizes GLUT4 transporters 320 to the muscle cell membrane, allowing glucose to leave the blood stream and enter the muscle cell, thus reducing a patient's blood glucose level.

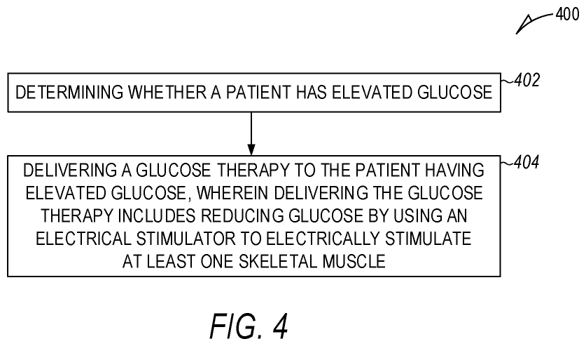

FIG. 4 illustrates, by way of example, a method for glucose control using skeletal muscle stimulation. The method 400 includes determining whether a patient has elevated glucose, at 402. A glucose therapy is delivered at 404, where delivering the glucose therapy includes reducing glucose by using an electrical stimulator to electrically stimulate at least one skeletal muscle.

In various embodiments, the skeletal muscle stimulation for glucose therapy is controlled by the patient. In other embodiments, the skeletal muscle stimulation for glucose therapy is automated. The stimulation device is paired with one or more physiological sensors, in various embodiments, to turn on or send an alert to the patient to initiate therapy when blood glucose levels are high, and to turn off or notify the patient to stop stimulation when blood glucose levels are normal, have reached the target level, or are low. In addition, a physiological sensor can capture blood glucose levels to track patient condition and therapeutic efficacy. Additional physiological signals that can be monitored include, but are not limited to: transcutaneous monitoring of temperature, edema and electromyography (EMG). Additional biomarkers can be used as an indication of skeletal stimulation level and/or GLUT4 or glucose levels, including tissue impedance, glycogen, and measures of therapeutic efficacy such as HbA1c and markers of microvascular or macrovascular complications. Other biomarkers such as metabolites and markers of insulin resistance can be used to provide feedback or for therapy initiation.

In various embodiments, motor nerves can be stimulated instead of muscle, which may allow for capture of greater muscle mass or use less energy per contraction. In some embodiments, a clock, activity sensor, or sleep sensor may be used to prevent delivering stimulation when it would be particularly bothersome to the patient, such as when sleeping or attempting to fall asleep. The therapy can be scheduled, based on physical condition of the patient, activity level of the patient, brady condition of the patient, time of day, pregnancy or based on treatments for other conditions of the patient.

In various embodiments, the electrical stimulation is delivered to stimulate the muscle, when blood glucose level is high, to burn energy to remove glucose from the blood, using stimulation parameters selected to cause the at least one skeletal muscle to contract without patient discomfort. In addition, stimulation can be delivered to sensory nerves to block or reduce the sensation caused by muscle/motor nerve stimulation. The battery or power supply of the pulse generator is rechargeable via wired and/or wireless charging, in some embodiments. In some embodiments, the battery or power supply of the pulse generator is replaceable.

The system can stimulate skeletal muscles using a current of 1 to 50 mA and a frequency of 0.5 to 100 Hz, in various embodiments. The therapy targets 50% or more recruitment of muscle fibers in a limb region, in an embodiment. In various embodiments, therapy is applied such that the current does not trigger strong contractions but still facilitates glucose transport, to reduce the sensation caused to the patient. Physiological parameters, such as force of muscle contraction through EMG quantification (raw electrical activity, amplitude, or integrated EMG) can be used to assist in parameter selection as well as with adapting existing stimulation parameters, in various embodiments. Thus, input signals from sensors can be used to update stimulation parameters to achieve the desired magnitude of electric field and muscle response, avoiding severe contractions that would lead to patient discomfort. In various embodiments, the skeletal muscle stimulation system may be worn or turned on intermittently by the patient. For example, the patient can wear the device for a limited time period, such as for one hour, to simulate exercising for the limited time period. In various embodiments, the stimulation and/or blocking therapy may be delivered in the form of electrical energy, magnetic energy, sound energy (e.g., ultrasound), light energy (e.g., laser energy, infrared energy, or photodynamic therapy) and/or heat energy. Other types of stimulation therapy can be delivered to stimulate skeletal muscles without departing from the scope of the present subject matter.

In various embodiments, the present skeletal muscle glucose therapy can be used as a standalone therapy. In some embodiments, the present skeletal muscle glucose therapy can be combined with other types of glucose therapy, such as an insulin pump, oral insulin-releasing drugs such as dipeptidyl peptidase 4 (DPP4) inhibitors or Sulfonylureas, injectable insulin, spinal cord stimulation, dorsal root ganglia stimulation, portal vein modulation and/or hepatic artery modulation (a.k.a. non-skeletal muscle glucose therapies). Many non-skeletal muscle glucose therapies are diabetic therapies uses to treat diabetes in diabetic patients. Non-skeletal muscle glucose therapies can be combined with the present skeletal muscle glucose therapy without departing from the scope of the present subject matter.

In an embodiment, a non-skeletal muscle glucose therapy (e.g. insulin pump) is a primary therapy and the skeletal muscle glucose therapy is a secondary therapy, where the secondary therapy is enabled as needed to improve the effectiveness of glucose therapy or reduce a dose of the primary therapy. In another embodiment the skeletal muscle glucose therapy is a primary therapy and a non-skeletal muscle glucose therapy (e.g. insulin pump) is a secondary therapy, where the secondary therapy is enabled as needed to improve the effectiveness of glucose therapy or reduce a dose of the primary therapy. In yet another embodiment, the skeletal muscle glucose therapy is enabled during one patient condition (e.g. wakefulness, inactivity) and a non-skeletal muscle glucose therapy is enabled during another patient condition (e.g. sleep, activity).

Various motor nerves and targeted vessels can be used to deliver the present skeletal muscle glucose therapy. Motor nerves can include fiber class A, subclass alpha (efferent to muscles), motor neurons, subclass gamma (efferent to muscle spindles), and muscle tone. When stimulating leg muscle in the thigh region, target nerves may include a femoral nerve (a major nerve that serves the tissue of the thigh and leg, including muscles and skin), a sciatic nerve, a saphenous nerve, a posterior femoral cutaneous nerve, gluteal nerves and/or obturator nerves. Vessels that can be used for transvascular stimulation in the thigh area may include a femoral artery and its branches, including a deep femoral artery, a lateral circumflex femoral artery, a medial circumflex femoral artery, a saphenous vein, and/or a femoral vein.

In various embodiments, initial titration of the present skeletal muscle stimulation therapy can be performed in an office of a medical provider with EMG electrodes to measure magnitude of response. An option for at-home titration, subsequent to the initial office visit, may include a wearable strap that goes around the thigh which includes multiple EMG sensors, and is communicatively coupled to the stimulation device and/or patient controller. In one example, a voluntary contraction is measured and stimulation parameters are then set to obtain below 50% of the magnitude of the voluntary contraction. The percent activation of skeletal muscle could be based on patient preference and tolerance and could be an excess of 50% in some embodiments. Alternatively, although less precise, patients may provide subjective feedback on the percent muscle contraction based on their perception to guide therapy settings.

In various embodiments, a frequency assessment is performed to determine at which frequency patient experiences tonic contraction, and then the frequency is reduced below the threshold with appropriate safety margin. In one example, the in-office characterization of stimulation frequency/amplitude-to-EMG response can be used to establish a baseline. This baseline can then be used to establish and characterize various stimulation programs, leaving the selection of programs in everyday use to the patient. In some embodiments, the patient has an interface with an on/off button to control the therapy if parameters/stimulation were inappropriate or uncomfortable. In other embodiments, feedback physiological signals can provide an indication of stimulation strength. In some examples, additional measures can be used to assist with in-office or at-home titration, such as lactic acid, mechanical measures such as a strain sensor, either implanted or externally worn, around the leg, and/or tissue impedance measures to obtain appropriate electric field/voltage settings.

Overall stimulation parameters can be varied to achieve varying levels of stimulation and therapeutic effects, in various embodiments. Stimulation levels can vary between low (e.g., sub-perception, low-perception), medium, and high settings which can result in varying degrees of glucose uptake. For example, during stimulation, glucose uptake is enhanced through contraction-stimulated glucose uptake (which occurs quickly during the therapy session) along with post-exercise increase in insulin sensitivity (which occurs post-exercise and last several hours). The high intensity stimulation may provide the strongest post-exercise/post-session effect, altering metabolism and increasing insulin sensitivity. In one example, to achieve small individual contractions (low frequency stimulation; 0.5-100 Hz) as opposed to tonic contraction (high frequency stimulation), a preferable frequency range for muscle stimulation is 1-20 Hz. Stimulation at high frequencies, e.g. greater than 100 Hz, can result in neuromuscular junction depletion and therefore reduced muscle contractions and lower efficacy.

In various embodiments, monophasic or biphasic direct current (DC) pulses may be used. Biphasic DC pulses avoid charge accumulation which could damage tissue, in various embodiments. In various embodiments, varying the amplitude of the electrical stimulation of skeletal muscle will vary the number of motor units recruited in the contraction. Amplitude may be set individually for each patient and can depend on electrode location and configuration. Constant or variable amplitude may be used to recruit muscle, in some examples. In one embodiment, biological feedback signals may assist in determining optimal thresholds and percentage of muscle contraction.

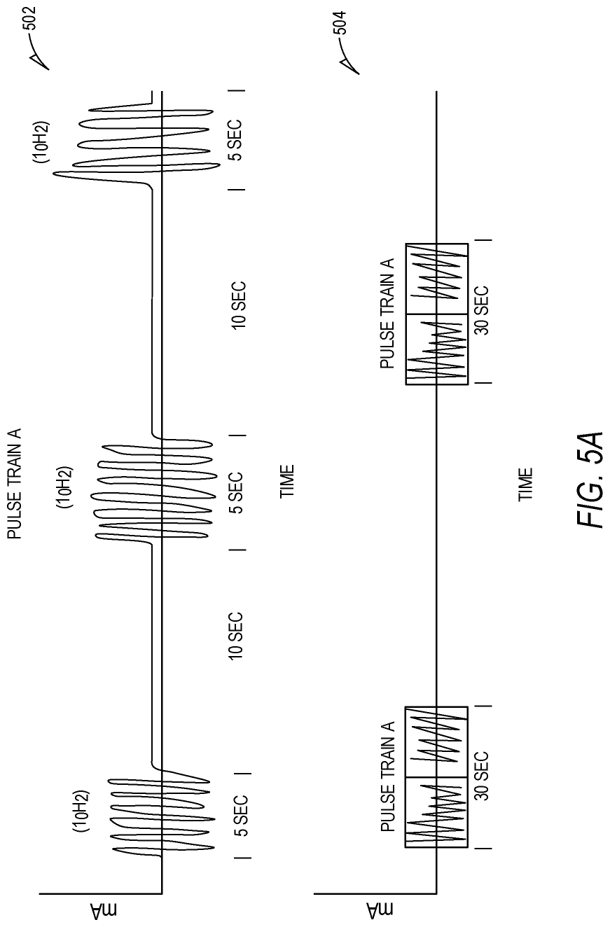

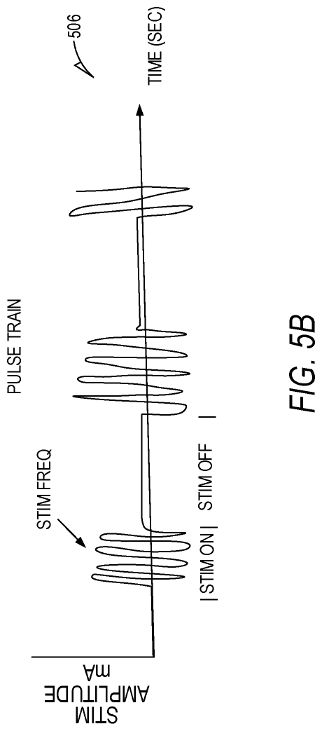

FIGS. 5A-5B illustrate, by way of example, graphical representations of electrical stimulation signals used for skeletal muscle stimulation for glucose control. Various duty cycles (time on/time off) of applied therapy may be used to tailor the stimulation therapy to patient preference. In some embodiments, continuous stimulation may be used. In some embodiments, pulse train stimulation may be used. In still further embodiments, a combination of continuous and pulse train stimulation may be used. For example, a patient may receive continuous stimulation of 1 Hz for 15 minutes at an amplitude of 50% of voluntary contraction level. Alternatively, various pulse trains may be used to more maximally exercise muscles between contracted and non-contracted states. In some examples, the duty cycle may be varied to prevent adverse effects or prevent fatigue in the patients. In some embodiments, the amplitude, frequency, pulse shape and/or pulse train may be continuously variable, for example, to represent variation in regular exercise.

FIG. 5A illustrates an example stimulation protocol for medium intensity skeletal muscle stimulation for glucose control. At 502, a pulse train is provided using 10 Hz stimulation with a duty cycle of 5 seconds on and 10 seconds off, at an amplitude equivalent to 25%-50% of voluntary contraction. At 504, a therapy setting is provided where this pulse train is delivered in 30-minute intervals (“ON”) with a duration of 1+ hours (“OFF”) between “ON” intervals. Further examples can be used for higher or lower intensity stimulation, in various embodiments. For a high intensity therapy protocol, amplitude may be increased to >50% of voluntary contraction level, or a percentage of voluntary contraction level as selected by a patient or physician. In various embodiments, the patient may choose a “high intensity”, “medium intensity”, and/or “low intensity” setting based on the patient's preference. In some embodiments, duration of “therapy OFF” may be greater than with the low intensity or medium intensity settings, e.g. 12-24 hours. For a low intensity therapy protocol, amplitude may be reduced to <25% of voluntary contraction level. This setting may particularly be useful when patients are resting or sleeping, keeping contraction level to a low perception level.

The previously described therapy levels are an example, and the present skeletal stimulation therapy can be provided at a plurality of levels, frequencies, pulse trains and/or therapy settings. More generically, therapy parameter settings may include:
i. Stimulation Frequency: 0.5 to 100 Hz
ii. Stimulation ON time: 0.5 to 300 seconds
iii. Stimulation OFF time: >0.5 seconds
iv. Stimulation Amplitude: 5%-75% of maximum voluntary contraction level

FIG. 5B illustrates an example stimulation protocol using varying parameters for skeletal muscle stimulation for glucose control. Instead of using fixed values as shown above, it may be beneficial for the parameters to vary within a therapy session (similar to spinal cord stimulation (SCS) for pain arbitrary waveforms, which involve some amount of varying frequencies, for example). In the example of this skeletal muscle stimulation embodiment 506, varying stimulation parameters may more closely mimic a true exercise regimen, for example.

Various embodiments may use electrode impedance measurements to optimize electrode selection and monitor tissue. The medical device may be an implantable pulse generator configured to stimulate skeletal muscle to promote glucose uptake. The medical device may be used to deliver a therapy for any condition requiring the regulation of blood glucose levels. For example, the therapy may treat diabetes. Other conditions that may be treated may include insulin resistance, genetic metabolic disease, hyperglycemia, obesity, hyperlipidemia, hypertension, endocrine diseases and/or inflammatory disorders.

The stimulation and/or blocking therapy may be delivered in the form of electrical energy, magnetic energy, sound energy (e.g. ultrasound), light energy (e.g. laser energy, infrared energy, etc. including photodynamic therapy) and/or heat energy, amongst other modalities.

The stimulation may be in a form of stimulation pulses that are characterized by pulse amplitude, pulse width, stimulation frequency, duration, on-off cycle, pulse shape or waveform, temporal pattern of the stimulation, among other stimulation parameters. Examples of the stimulation pattern may include burst stimulation with substantially identical inter-pulse intervals, or ramp stimulation with incremental inter-pulse intervals or with decremental inter-pulse intervals. In some examples, the frequency or the pulse width may change from pulse to pulse.

Systems and methods, according to various embodiments, have been described. Some specific examples of therapies that may be implemented using the described system and methods are provided below.

Various embodiments may control delivery of electrical energy using patient input such as dietary intake, mealtime, exercise or patient-activated therapy session. Various embodiments include an activity sensor for use to detect exercise levels, and automatically, semi-automatically or manually control therapy delivery using the detected exercise levels.

The sensor(s) may include at least one of an optical, electrochemical, biopotential, impedance, or electromagnetic sensor. The sensor may be an implantable sensor, or a partially invasive device such as an external sensor with microneedles penetrating the skin. The sensor may be a non-invasive sensor. The system may include a patient interface that allows the patient to control therapy parameters, and may incorporate patient progress over time including at least one of a glucose metric trend, therapy usage trend, and activity level trend.

Some embodiments may provide a system including a passive implanted lead including both a sensor providing input to a handheld patient controller (e.g. patient instruction to begin therapy) or providing input to a wearable pulse generator. The system may comprise an external pulse generator configured with a transmitter capable of transmitting waveform parameters and electrical energy across tissue to an implanted receiver, an implantable lead including at least one electrode at a distal end and a receiver at a proximal end capable of receiving the waveform parameters and electrical energy from the transmitter. The system may include a sensor configured to sense at least one parameter from the patient indicative of a glucose level, a processor configured to receive an input from the sensor and generate a recommended therapy setting, and a controller that may be activated by a patient to cause the external pulse generator to initiate, change or stop therapy to modulate glucose levels.

Some embodiments may include a patient interface. The patient interface may include a glucose measurement and a recommended therapy session. The patient interface may allow a patient to input dietary intake and mealtime, which may be used by the processor to calculate the recommended therapy session. Various embodiments may include a patient-facing or physician-facing interface that trends patient progress over time, including at least one of a glucose metric trend, therapy usage trend, and activity level trend.

Various embodiments disclosed above include a glucose monitor. Any direct or indirect measure of glucose may be used in our closed-loop system, either via a sensor communicatively coupled to a processor and controller within a pulse generator, or to a processor within a non-invasive device such as a handheld remote/patient interface. For examples of an indirect measures, enzymes can be measured, including glucose-6-phosphatase, glucose oxidase, pyruvate, fructose 1,6 biphosphate, phosphoenolpyruvate, glyceraldehyde 3-phosphate, phosphofructokinase, and glycated hemoglobin. By way of example, Vaddiraju et al., J Diabetes Sci Technol. 2010 November; 4(6): 1540-1562, refer to a number of continuous glucose monitoring technologies. Vaddiraju et al. is incorporated herein by reference, as these glucose monitoring technologies may be used in the present subject matter.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
delivering a glucose therapy to a patient having elevated glucose, wherein delivering the glucose therapy includes reducing glucose using an electrical stimulator to electrically stimulate at least one skeletal muscle.

2. The method according to claim 1, further comprising:
receiving a signal from a sensor configured to sense a level of glucose or a surrogate of glucose of a patient; and
titrating the glucose therapy using the signal.

3. The method according to claim 1, wherein the glucose therapy further includes delivering insulin from an insulin pump.

4. The method according to claim 1, wherein the glucose therapy further includes ingesting oral insulin-releasing drugs.

5. The method according to claim 1, wherein the glucose therapy further includes percutaneously injecting insulin.

6. The method according to claim 1, further comprising delivering the electrical stimulation in combination with a spinal cord stimulation.

7. The method according to claim 6, wherein the spinal cord stimulation includes therapy for pain reduction.

8. The method according to claim 1, further comprising delivering the electrical stimulation in combination with dorsal root ganglia stimulation.

9. The method according to claim 1, further comprising delivering the electrical stimulation in combination with stimulation of targets in and around a portal vein.

10. The method according to claim 1, further comprising delivering the electrical stimulation in combination with stimulation of targets in and around a hepatic artery.

11. A non-transitory machine-readable medium including instructions, which when executed by a machine operably connected to electrodes, cause the machine to:
deliver a glucose therapy to a patient having elevated glucose, including reducing glucose using an electrical stimulator to electrically stimulate at least one skeletal muscle.

12. The non-transitory machine-readable medium according to claim 11, wherein the instructions further cause the machine to:
receive a signal from a sensor configured to sense a level of glucose or a surrogate of glucose of a patient; and
titrate the glucose therapy using the signal.

13. The non-transitory machine-readable medium according to claim 11, wherein the instructions cause the machine to deliver electrical stimulation to one or more of a femoral nerve, a sciatic nerve, a saphenous nerve, a posterior femoral cutaneous nerve, a gluteal nerve, and an obturator nerve.

14. The non-transitory machine-readable medium according to claim 11, wherein at least one of the electrodes is within a blood vessel to deliver electrical stimulation transvascularly.

15. The non-transitory machine-readable medium according to claim 14, wherein at least one of the electrodes is within at least one of a femoral artery, a saphenous vein, or a femoral vein.

16. The non-transitory machine-readable medium according to claim 11, wherein the instructions cause the machine to titrate the glucose therapy based on anticipated or scheduled dietary intake, anticipated or scheduled activity, or location of the patient.

17. The non-transitory machine-readable medium according to claim 11, wherein the instructions cause the machine to titrate the glucose therapy based on a received input from the patient.

18. The non-transitory machine-readable medium according to claim 11, wherein an intensity of the electrical stimulation is increased to increase a rate of glucose reduction.

19. The non-transitory machine-readable medium according to claim 11, wherein the instructions cause the machine to deliver the glucose therapy on a schedule.

20. The non-transitory machine-readable medium according to claim 11, wherein the electrical stimulation is delivered using stimulation parameters selected to cause the at least one skeletal muscle to contract without causing discomfort in the patient.
